(19) United States
(12) Patent Application Publication
(10) Pub. No.: US 2025/0111911 A1
(43) Pub. Date: Apr. 3, 2025

(54) SYSTEM FOR DETERMINING AND RENDERING BIOMETRIC TRENDS ACROSS PRIOR STUDIES AND METHOD OF OPERATION THEREOF

(71) Applicant: KONINKLIJKE PHILIPS N.V., EINDHOVEN (NL)

(72) Inventors: Jon Kamerman, Andover, MA (US); Richard Earl Gregg, Cambridge, MA (US); Jeff J Wei, Shanghai (CN)

(21) Appl. No.: 18/805,627

(22) Filed: Aug. 15, 2024

(30) Foreign Application Priority Data
Aug. 17, 2023 (WO) PCT/CN2023/113460
Aug. 5, 2024 (WO) PCT/EP2024/072124

(51) Int. Cl. G16H 10/60; G16H 50/20

(52) U.S. Cl. CPC G16H 10/60; G16H 50/20

(57) ABSTRACT

A clinical monitoring system for determining trends across medical studies, includes a controller configured to generate links, each link associating a corresponding data point (DP) of DPs with a medical record of medical records from which the corresponding DP was derived; render a medical viewing environment (VE) for a current diagnostic type, the VE including at least one link associated with trend information; determine whether the link associated with the trend information has been selected; open a trend environment comprising the trend information and having at least one trend graph comprising a plurality of corresponding datapoints (DPs) of the plurality of data points (DPs) when it is determined that the link associated with the trend information has been selected; determine whether a DP of the DPs is selected in the trend graph; and render the medical record associated with the selected DP by a corresponding link.

### FIELD OF THE PRESENT SYSTEM

The present system relates to a system for rendering biometric trends across prior medical studies and, more particularly, a system for accessing prior medical studies for a target patient, determining trends in these studies, and rendering results of the determination, and a method of operation thereof.

### BACKGROUND OF THE PRESENT SYSTEM

Unfortunately, without proper and timely medical care, a disease may progress undetected which can lead to a less than desirable outcome for a patient. Often, changes in diagnostic biometric measurements over time may indicate progress of a disease. Currently clinicians need to manually synthesize data from prior diagnostic medical studies (DMSs), of which there may be many types (for example, Ultrasound, electrocardiogramputed tomography (CT), magnetic resonance imaging (MRI), Interventional Catheterization Laboratory (Cath Lab or (ICL)), Pathology Laboratory Analyzer (Pathology), etc.), before any attempt can be made at diagnosis which can be difficult to perform and is time consuming. For example, in order to view diagnostic data, the clinician often has to open specific medical applications, log in, open specific diagnostic toolsets, and find relevant DMSs separately for each of these medical applications. This process may be difficult if not impossible to perform in certain settings (e.g., in an emergency room (ER)), may be inconvenient, may be time consuming, may be prone to error, and may increase costs. Accordingly, prior art methods did not provide easy access to DMSs or easy synthesis of these DMSs.

Accordingly, embodiments of the present system provide features and advantages which obviate problems and difficulties of conventional electronic medical records (EMR) systems and methods. To overcome the aforementioned barriers and detriments as well as others, there is a need for a system which can reliably and inexpensively provide an accurate and fast system for rendering biometric trends across prior medical studies thus overcoming the aforementioned barriers and detriments as well as others of conventional systems.

### SUMMARY OF THE PRESENT SYSTEM

The system(s), device(s), method(s), arrangements(s), interface(s), computer program(s), processes, etc., (hereinafter each of which will be referred to as system, unless the context indicates otherwise), described herein address problems in prior art systems.

In accordance with embodiments of the present system, there is disclosed a clinical monitoring system for determining trends across medical studies, includes a controller configured to generate links, each link associating a corresponding data point (DP) of DPs with a medical record of medical records from which the corresponding DP was derived; render a medical viewing environment (VE) for a current diagnostic type, the VE including at least one link associated with trend information; determine whether the link associated with the trend information has been selected; open a trend environment comprising the trend information and having at least one trend graph comprising a plurality of corresponding datapoints (DPs) of the plurality of data points (DPs) when it is determined that the link associated with the trend information has been selected; determine whether a DP of the DPs is selected in the trend graph; and render the medical record associated with the selected DP by a corresponding link. The current viewing environment comprises a diagnostic viewing environment (VE) or worklist/inbox (WI) viewing environment (VE) for a current diagnostic type. The diagnostic type may be selected from one of an Echocardiogram (ECG) scan, an Ultrasound scan, a Computed Tomography (CT) scan, a Magnetic Resonance Imaging (MRI) scan, and Interventional Catheterization Laboratory (Cath Lab or (ICL)) report, and a Pathology Laboratory Analyzer (Pathology) report.

The controller may be further configured to obtain the plurality of medical records in accordance with system setting information (SSI) from an electronic medical record (EMR) database; obtain the plurality of medical records associated with a selected patient; extract each of the plurality of data points (DPs) over a period of time in accordance with the system setting information (SSI). The controller may be further configured to include a reverse link in the rendered medical record, the reverse link associating the selected data point (DP) and the medical record from which the selected data point (DP) was derived; determine whether the reverse link was selected, and return to the trend environment when it is determined that the reverse link was selected; and determine whether hovering is performed over a data point (DP) of the plurality of data points (DPs) and render a value of the corresponding data point (DP) of the plurality of data points (DPs) when it is determined that hovering is performed over a data point (DP).

### BRIEF DESCRIPTION OF THE DRAWINGS

It should be expressly understood that the drawings are included for illustrative purposes and do not represent the scope of the present system. It is to be understood that the figures may not be drawn to scale. Further, the relation between objects in a figure may not be to scale and may in fact have a reverse relationship as to size. The figures are intended to bring understanding and clarity to the structure of each object shown, and thus, some features may be exaggerated in order to illustrate a specific feature of a structure. In the accompanying drawings, like reference numbers in different drawings may designate identical or similar elements, portions of similar elements and/or elements with similar functionality. The present system is explained in further detail, and by way of example, with reference to the accompanying drawings which show features of various exemplary embodiments that may be combinable and/or severable wherein:

FIG. 1 illustratively shows a schematic block diagram of a portion of a system operating in accordance with embodiments of the present system;

FIGS. 2A-2C are an illustration including portions of a diagnostic ECG viewing environments, and s trending environment including a trend view (TV) graph generated in accordance with embodiments of the present system;

FIG. 3 is an illustration which shows a functional flow diagram performed by a process in accordance with embodiments of the present system;

FIG. 4 is an illustration of a portion of the diagnostic viewing environment of FIGS. 2A-2C in accordance with embodiments of the present system;

FIG. 5 is an illustration including portions of viewing environment and a trend view graph generated in accordance with embodiments of the present system;

FIGS. 6A-6B are an illustration including portions of a diagnostic ECG viewing environment and trending environment including a trend view (TV) graph generated in accordance with embodiments of the present system;

FIG. 7 is an illustration including portions of a diagnostic ECG viewing environment and trending environment including a trend view (TV) graph arranged in a side-by-side orientation generated in accordance with embodiments of the present system; and

FIG. 8 is an illustration including portions of a diagnostic ECG viewing environment and trending environment including a trend view (TV) graph arranged in a stacked view orientation generated in accordance with embodiments of the present system.

### DETAILED DESCRIPTION OF THE PRESENT SYSTEM

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well-known devices, circuits, tools, techniques and methods are omitted so as not to obscure the description of the present system.

The term “and/or,” and formatives thereof, should be understood to mean that only one or more of the recited elements may need to be suitably present (e.g., only one recited element is present, two of the recited elements may be present, etc., up to all of the recited elements may be present) in a system in accordance with the claims recitation and in accordance with one or more embodiments of the present system. In the context of the present embodiments, the terms “about”, substantially and “approximately” denote an interval of accuracy that a person skilled in the art will understand to still ensure the technical effect of the feature in question which in some cases may also denote “within engineering tolerances.” For example, the terms may indicate a “deviation” from indicated numerical value(s) of ±20%, ±15%, ±10%, ±5%, ±1%±0.5% or ±0.1%.

The terms clinician, clinicians, or formatives thereof, may include a professional such as a doctor, a clinician, a technologist, an operator, an expert, a nurse, a medical specialist, a technician, a prescriber, and/or the like, who may operate and/or review information related to the patient such as system setting information (SSI), system information, and/or data generated by embodiments of the present system and associated data for review by a clinician, an expert, a medical specialist, a doctor, an operator, a technician, and/or the like unless the context indicates otherwise. The terms patient, patients, or formatives thereof, may include patients or other individuals (e.g., persons, subjects, subjects under test, etc.) or other users who may be receiving any type of monitoring or assessment using systems, machines, processes, and/or methods operating in accordance with embodiments of the present system.

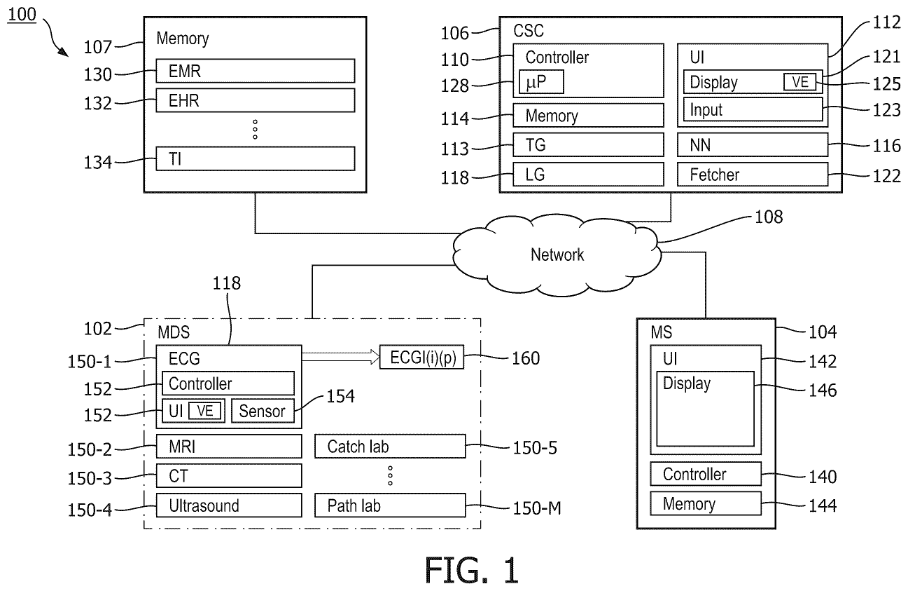

FIG. 1 illustratively shows a schematic block diagram of a portion of a system 100 (hereinafter the system 100) operating in accordance with embodiments of the present system. The system 100 may include one or more of a medical diagnostic system (MDS) 102 which includes at least one medical diagnostic devices (MDD) 150-X, a mobile station (MS) 104, a clinical service center (CSC) 106, and a memory 107, one or more of which may communicate with the other via a network 108 using any suitable communication method or methods such as wired and/or wireless (e.g., optical) communication methods over the network 108.

The network 108 may include any suitable network such as an ad-hoc network, a body area network (BAN), a personal area network (e.g., e.g., Bluetooth™, etc.), a Zigbee network, Wi-Fi, a local area network (LAN), a wide area network (WAN), a telephony network, a cellular network (e.g., 5G, etc.), a controller area network (CAN), etc. In some embodiments, the network 108 may include any suitable network or networks which may enable connectivity between portions of the system 100. In some embodiments, the network 108 may operate in accordance with one or more standards which may provide for medical device connectivity (e.g., as may be defined by EN ISO/IEEE 11073 Health informatics standards, etc.). This may enable interoperability between connected medical devices within the system 100 and/or other portions of the system 100 such as the memory 107, the CSC 106, etc.

The MDS 102 may include one or more medical diagnostic devices (MDDs) 105-X, such as an ECG 150-1, and MRI 150-2, a CT scanner 150-3, and Ultrasound scanner 150-4, and ICL 150-5, and a Path Lab 150-M. In the present embodiments, the MDS may include M MDDs where M is an integer.

The MDS 102, the MS 104, the CSC 106, the memory 107, and the network 108 may be controlled by at least one controller (e.g., one or more of controllers 110, 152, 140, etc.) which may be local or distributed and may which may control the overall operation of the system. The at least one controller may include one or more logic devices such as a microprocessor and/or the like and may operate using distributed computing methods.

The CSC 106 may include one or more of a controller 110, a memory 114, a user interface (UI) 112, a neural network (NN) 116, a link generator (LG) 118, a fetcher 122, and a trend generator (TG) 113, one or more of which may communicate with the other. The controller 110 may control the overall operation of the CSC 106 and, as such, may receive information from one or more memories of the present system (e.g., the memory 107, 114, etc.) for further processing. For example, the controller 110 may control the fetcher 122 to fetch desired information such as one or more desired electronic health records (EHRs), etc., for a desired patient from the memory 107 using any suitable method or methods. For the sake of clarity, it will be assumed that the EMR 130 may include the EHR 132 as well as any other third party applications or databases (DBs) (such as a hospital information system (HIS), a picture archiving and communication system (PACS), etc.) that may store medical records of the patient that may be employed by embodiments of the present system. The CSC 106 may communicate with the memory 107, the MDS 102 or at least one of the MDD's 150-X, and/or MS 104 to, for example, transmit diagnostic results of the processing (e.g., trend information (TI), etc.) to be rendered on a rendering device of the system such as on a rendering device of the system (e.g., a display) for the convenience of the clinician. This rendering device may, for example, include a display of the MDS 102 or at least one of the MDD's 150-X, a display of the MS 104, and/or a display of the CSC 106 as may be desired. The results of the system processing EMRs may include one or more of TI, link information (LI), patient information (PI), diagnostic results (DRs), and the like and may be stored in any suitable memory of the system such as in the memory 107 as diagnostic information (DI) 134. The CSC 106 may process data in real time or may obtain data that has been stored in a memory of the system such as from the memory 107, 114, and/or 144. For example, the CSC 106 may request information (e.g., via a fetch operation) such as electronic medical records (EMRs), electronic health records (EHRs), and/or diagnostic information (DI) (e.g., each for one or more patients if desired) from one or more the EMR memory 130, the EHR 132, and/or the DI 134, respectively, and may receive the desired information via the network 108.

The UI 112 may include a rendering device such as a display 121, a speaker, etc., and a user input interface 123 with which a clinician (or other user) may interact to enter information such as a touchscreen, etc.

The controller 110 may be operable for providing control signals and/or performing operations in response to input signals from the user input interface 123 as well as in response to other devices of a network, such as the MDS 102, the MS 104, etc., and executing instructions which may be stored in, and/or retrieved from, a memory of the system such as the memory 114, 107 and/or 144. The controller 110 may include one or more of a microprocessor (e.g., a μP 128), an application-specific and/or general-use integrated circuit(s), a logic device, etc. It is envisioned that the μP 128 may be a dedicated processor for performing in accordance with the present system and/or may be a general-purpose processor wherein only one of many functions operates for performing in accordance with embodiments of the present system. The μP 128 may operate utilizing a program portion, multiple program segments, and/or may be a hardware device utilizing a dedicated and/or multi-purpose integrated circuit.

It is envisioned that one or more portions of the CSC 106, such as the controller 110, may be operationally coupled to one of more of the memory 114, the UI 112, the neural network 116, the link generator (LG) 118, the fetcher 122, and the trend generator (TG) 113. The memory 114 may include any suitable memory configured to store information used by, and/or generated by, the CSC 106, such as operating instructions, the SSI, the SMU, PI, LI, TI, DI, etc. In some embodiments, the memory 114 may store information related to applications, diagnostic test types (e.g., ECG, CT-scan, MRI, etc.), operation flow, etc.

The UI 112 may include a display 121 (e.g., a touch-screen display, etc.) and a user input interface 123 (e.g., a hard or soft key interface, etc.) which may be combined with, or separate from, each other. For example, in some embodiments, the user input interface 123 may include a keyboard, a mouse, a trackball, touch sensitive sensor, and/or other device, such as a touch-sensitive display, which may be located locally or remotely from one or more portions of the CSC 106. For example, the user input interface may be stand alone or part of a system, such as part of a laptop, a personal digital assistant (PDA), a mobile phone (e.g., a smart phone), a smart watch, an e-reader, a monitor, a smart or dumb terminal, the MS 104, and/or other device for communicating with the controller 110 via any operable link such as a wired and/or wireless communication link (e.g., via the network 108, etc.). The user input interface 123 may be operable for interacting with the controller 110 including enabling interaction within a UI 112 as described herein. Clearly the controller 110, the UI 112, the memory 114, the MS 104, the MDS 102, and the network 108 may all or partly be a portion of a computer system or other device. The UIs such as the UI 112 may be operative to provide audio/visual feedback as well as graphical user interfaces (GUIs) to the clinician (e.g., the present user) of the present system and may inform the clinician of information generated by the present system, operating parameters, operating states, etc. and may be operative to receive information and/or instructions from the clinician. In some embodiments, the UI (such as the UI 112) may include one or more sensors or transducers with which a user may enter information such as a microphone with which the clinician may enter voice commands for the convenience of the clinician. These voice command may then be translated into operating instructions using any suitable methods of methods. Information corresponding to the voice commands may then be provided controller 110 which may translate these commands and may be operative to function accordingly. Similarly, the UI may include a haptic device and/or speaker with which information may be rendered for the convenience of the clinician.

The controller 110 may be operative to render content, such as still or video information, as well as audio information on a rendering device of the system such as on the display 121 and/or speaker of the UI 112. This information may include information related to operating parameters, instructions, feedback, links, browser windows (e.g., iFrames and the like), and/or other information related to an operation of the system, or portions thereof, such as SSI, system user information (SUI) such as including patent identification (ID), EHR, EMR, clinical decision support information (CDSI), link information (LI), patient information (PI), trend information (TI), diagnostic information (DI), application data, data generated by the system, and/or combinations thereof. The system 100 may analyze data generated by the system and may generate determinations such as CDSI, TI, etc., which may be stored in the system and/or rendered for the convenience of the patient and/or clinician depending upon settings in the SSI. Where SSI may be employed by the system so set operational parameters and settings and may be set by the system and/or user.

The neural network 116 may include any suitable neural network such as a stochastic neural network and/or other artificial neural network, etc. During operation, the neural network may receive as inputs such as EMRs corresponding to a selected patient and may output information related to trends such as trend information and corresponding links. Variances may be employed to mask or ignore certain measurements, when their removal may lead to better performance of an automated model.

The application data, SSI, etc., one or more models related to the neural network 116, and/or combinations thereof, may be received by the controller 110 for configuring (e.g., programming) the controller 110 to perform operation acts in accordance with embodiments of the present system. The controller 110 so configured becomes a special purpose machine particularly suited for performing in accordance with embodiments of the present system.

The methods of the present system are particularly suited to be carried out by a computer software program, such program containing modules corresponding to one or more of the individual steps or acts described and/or envisioned by the present system. Such program may of course be embodied in a computer-readable medium, such as an integrated chip, a peripheral device or memory, such as the memory 114, and/or other memory coupled to the controller 110.

The program and/or program portions contained in the memory 114 may configure the controller 110 to implement the methods, operational acts, and functions disclosed herein. The memories may be distributed, for example between MDD 102, the MS 104, the CSC 106, the memory 107, and the controller 120, where additional processors may be provided, may also be distributed or may be singular. The memories may be implemented as electrical, magnetic, and/or optical memory, or any combination of these or other types of storage devices. Moreover, the term “memory” should be construed broadly enough to encompass any information able to be read from or written to an address in an addressable space accessible by the μP 128 of the controller 110. With this definition, information accessible through a network, such as the network 108, is still within the memory, for instance, because the μP 128 may retrieve the information from the network 108 for operation in accordance with embodiments of the present system.

One or more controllers of the system 100 may employ machine learning and/or artificial intelligence (AI) engines to process information generated by the system, such as the EMR, as well as data provided to the system such as models and/or historical information and may form corresponding determined trend information (DTI) which may then be rendered and/or stored in a memory of the system for later processing. Further, the one or more controllers of the system 100 may be operative to detect for example, data trends and/or may form links associated with EMR and may render and/or store results in a memory for later retrieval and/or processing. The controller may further forward these results to a corresponding patient or clinician for their convenience as may be set forth in the SSI. The DTI may be accessed by the CSC 106, the MS 104, and/or the MDS 102 (or at least one of the MDD's 150-X) of the system for further review, processing, and/or analysis.

The at least one controller may access the memory and may obtain system setting information (SSI) which may include one or more of operating parameters (e.g., diagnostic information data type (e.g., ECG, CT, MRI, etc.), etc.), threshold values, warnings, display settings, user information, patient information such as patient identification (ID), content (e.g., video, audio, etc.), etc. The SSI may be set by the system and/or user and may be updated during use. The at least one controller may generate and render an interface with which the patient and/or clinician may, for example, interact with to, for example, set, reset, select, and/or adjust one or more settings of the SSI and/or enter information (e.g., ID, test type, settings, parameters (e.g., test day, date, test duration, etc.), results, etc.) which may be stored in the SSI in a memory of the system.

The MS 104 may include any suitable mobile station(s) such as a smart-phone (e.g., an iPhone™, a Galaxy Phone™, etc.), a personal digital assistant (PDA), a smart watch (e.g., an Apple™ Watch™, a Galaxy™ watch, etc.), a smart ring (e.g., generally a token) and/or the like, and may include a memory 144, at least one controller 140, a user interface (UI) 142, and a transceiver (Tx/Rx) configured to communicate with the network 108.

The UI 142 may provide an interface with which a clinician may interact, such interfaces may include, inter alia, a touch-screen display 146, an optical sensor such as a camera, a proximity sensor, a speaker, and/or a microphone. However, other user interfaces are also envisioned. The controller 140 may be operative to control the UI 142 to render information, such as content, etc., on a display and/or a speaker for the convenience of the patient, and may receive information entered by the user, such as selections, requests, commands, patient settings, etc., using any suitable input method such as gesture inputs, a touch input, voice input, etc. The memory 144 may store information for use by the system such as the SSI, DTI, ID, system settings, system parameters, operating parameters, and/or combinations thereof, as discussed herein. The patient identification (ID) may be employed to identify the patient and/or the corresponding test results.

The MS 104 may transmit information such as operating parameters, instructions, requests, commands (e.g., a start command, etc.), to the CSC 106 and/or to or at least one of the MDD's 150-X MDS 102, and may receive information generated by the MDS 102/MDD 105-X such as diagnostic scans, operating parameters, and/or combinations thereof, for further processing. The MS 104 may further receive and/or transmit DTI and/or ID, as discussed herein. Data transfer may occur on a periodic or non-periodic intervals as may be set forth in the SSI or requested by the system (e.g., or at least one of the MDD's 150-X of the MDD 102, the CSC 106, and/or the MS 104), patient, and/or clinician. For the sake of clarity, only a single MS 104 is discussed unless the context indicates otherwise. However, without limitation other MSs may be employed by the system. For example, a clinician such as a doctor may have an MS which may receive information such as SSI, DTI, etc., from other portions of the system for further review, processing, and/or storage in accordance with embodiments of the present system.

The MDS 102 may include one or more medical diagnostic scanners (MDDs) 150-1 through 150-M (generally MDDs 150-X) such as an electrocardiogramachine (ECG), a magnetic resonance imaging (MRI) scanner, a computed tomography (CT) scanner, an Ultrasound scanner (e.g., an Ultrasonography scanner), an interventional catheter lab (CATH LAB or ICL) scanner, and a Pathology Lab (Path Lab or Pathology), respectively, where M is an integer, which may output a corresponding output (e.g., ECG, MRI, CT, Ultrasound ICL scans, and a Pathology report, respectively) which may then be stored in a memory of the system such as the EMR 130 for later retrieval and/or analysis. For the sake of clarity, it is assumed that each of the MDDs 150-x is of a disparate (e.g., different) diagnostic type (DT) different from the other of the MDDs 150-x (e.g., ECG 150-1 as opposed to CT scan 150-3) and may output a record (e.g., a diagnostic record or medical record) having a corresponding diagnostic data type (DDT) (e.g., output scan, report, etc.). Thus, each of the MDDs 150-x may output a record having a corresponding DTT. For example, the ECG 150-1 may output an ECG, the MRI 150-2 may output an MRI, the CT 150-3 may output a CT, the ultrasound 150-4 may output an ultrasound (e.g., a sonogram), the ICL 150-5 may output an ICL, and the Pathology 150-M may output a pathology report, each of which may be stored in the EMR for the corresponding patient as a record (e.g., a medical record). Although MDDs 150-x of different DTs are shown, it should be understood that the EMR 130 may include EMRs of the same DT (e.g., MRIs) from two different MRI scanners. Each of these records may include detailed, patient level, physiological data, such as, anatomical measurements, day, date, time, and/or other measurements and may be stored (e.g., archived) in a memory such as the EMR 130 of the memory 107 for later retrieval and/or processing such as for processing to determine TI which may be stored with the corresponding records. Thus, TI may be stored in association with each patient's records. It is further envisioned that the stored records may be obtained from the memory (e.g., by the fetcher 122), and rendered on a UI of the system with the associated trend information for the convenience of the clinician. In some embodiments, it is envisioned that the TI may be generated in real time. For the sake of clarity, it will be assumed that the EMR 130 may be distributed and may include information from disparate medical storage systems such as a hospital information system (HIS), a picture archiving and communication system (PACS), and/or the like. Embodiments of the system may include information related to logging in to disparate systems to access desired information such as medical records, image files, etc. which may be desired by embodiments of the present system.

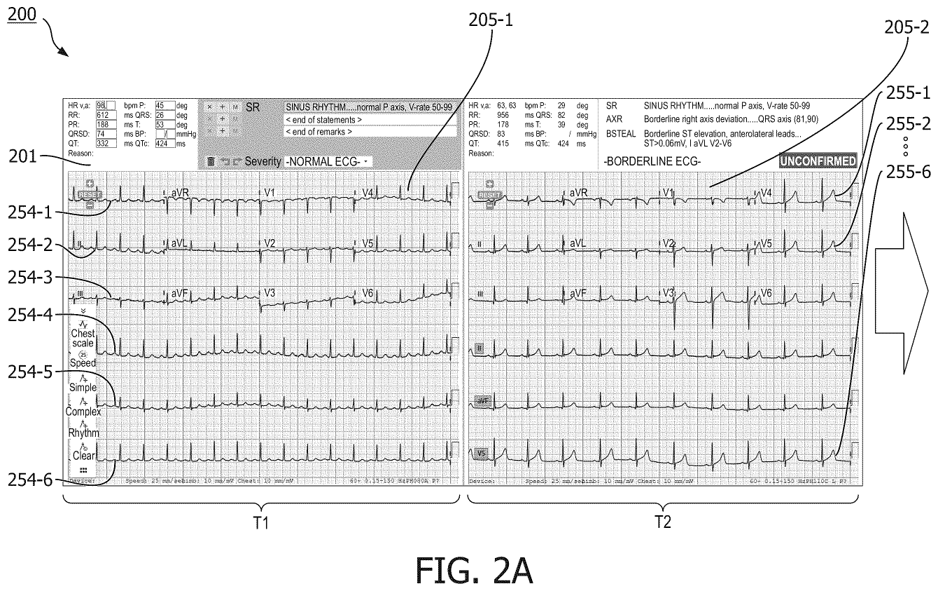

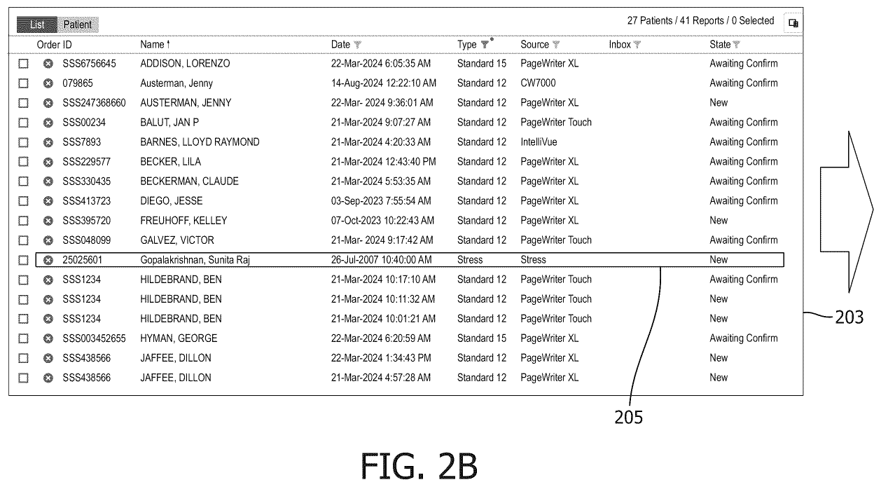

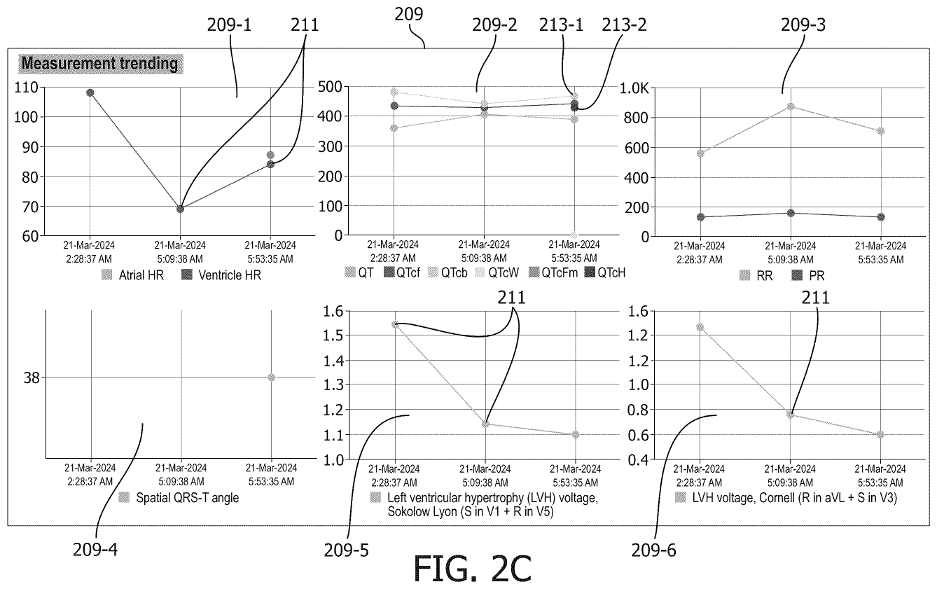

For the sake of clarity, the ECG 150-1 will now be discussed in more detail. However, it should be understood that other MDDs such as MDDs 150-2 through 150-M may include similar construction. Referring back to the MDD 150-1, this MDD may include a controller 152, a memory, an UI 152, a transmitter/receiver (Tx/Rx), and a plurality of sensors 154. The controller 152 may control the overall operation of the MDD 150-1 and may communicate with the network 108 via the Tx/Rx to, for example, transmit results of a scan (e.g., an ECG) to the EMR 130 for storage. One or more of sensors 154 may be coupled to the controller 120 using wired or wireless coupling and may be distributed throughout the system 100. Each of the one or more sensors 154 may sense related parameters, form corresponding sensor information, and provide this sensor information to the controller 152 for further processing such as to form a corresponding record (e.g., an ECG). It is envisioned that the one or more sensors 154 may be operative under the control of the controller 152 and may be configured to monitor the patient's physiology and form corresponding sensor information which may thereafter be processed (e.g., by the controller 152) to form a corresponding record (e.g., an ECG) that may be stored in a memory of the system such as in the EMR 132. The ECG may further be rendered in a viewing environment (VE) on a desired rendering device of the system such as on the UI (152). This is illustrated with reference to FIGS. 2A-2C which is an illustration 200 including portions of a diagnostic ECG viewing environments 201, 203, and a trending environment including a trend view (TV) graph 209 generated in accordance with embodiments of the present system. The ECG viewing environment (VE) 201 may include first and second EGC graphs 205-1 and 205-2 corresponding to ECG records (ECG scans) captured over two selected ranges of time (e.g., T1 and T2, respectively) and generated in accordance with embodiments of the present embodiments for a selected patient (e.g., the current patient). The SSI may further set forth selected ranges of time as well as desired sensor information (e.g., from desired sensors) for a viewing environment (e.g., ECG). Sensor information collected over ranges T1 and T2 for ECG sensors (leads) QT, QTcf, QTcb, QTcw, QTcFm, and QTcH are respectively graphically illustrated at lines 254-1 through 254-6 and 255-1 through 255-6 using corresponding line charts (or any other suitable graphical representation such as a bar chart) as may selected by the user and stored in the SSI. Data points (DPs) may be associated with each of the of the lines 254-1 through 254-6 and 255-1 through 255-6 of the graphs and may be linked to a trend viewing environment including trending information associated with one or more of the DPs. The trend view environment may include information related to trend information obtained from selected EMRs of the patient. The trending environment may include the trend view (TV) graph 209 which may be referred to as a trending view graph 209.

In some embodiments a worklist/inbox (WI) viewing environment (VE) may be rendered as illustrated in worklist/inbox graph 203. In this viewing environment, the system may obtain available EMR records 205 for the selected patient and which may be selected by the clinician and, in response, the process may retrieve the selected EMR record and render a viewing environment (VE) corresponding to information within the selected EMR record for the convenience of the user.

In either of these environments, the system may insert links which may be selected by the user (e.g., by selecting a highlighted area or a point of interest, a file name, etc., as may be set forth in the SSI) and which may be mapped to a corresponding trending environment including a trending view graph 209 may be generated and/or rendered as shown.

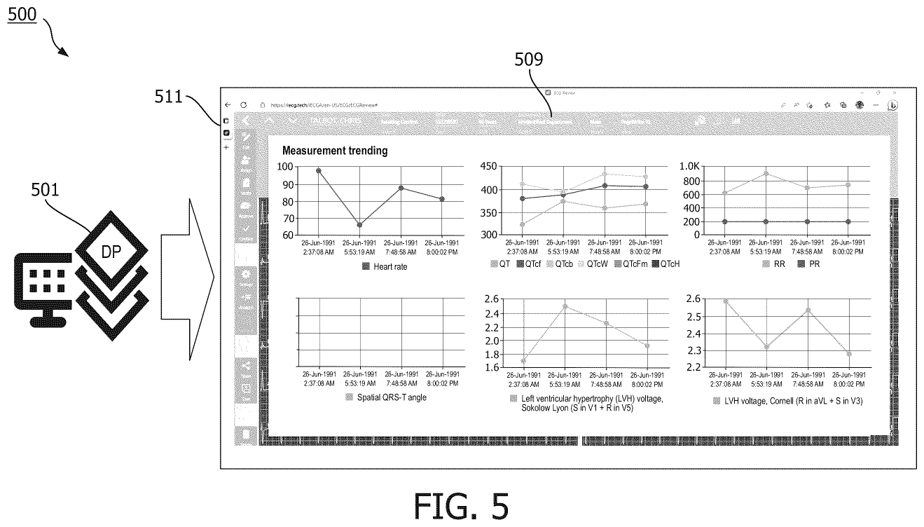

The trending environment as displayed by the trend view graph 209 may be launched from the Patient's diagnostic ECG viewing environment and/or the worklist/inbox viewing environment. However, in yet other embodiments is envisioned that the trend view graph 209 may be launched from a third party application as may be discussed with reference to FIG. 5.

It is assumed that the EMR 132 may include a plurality of electronic diagnostic records (e.g., medical records) of different DDTs the same DTT (e.g., ECGs, etc.) or of different DTTs (e.g., ECGs, MRIs, etc.). It is envisioned that the system 100 may monitor each of the patient's physiology over a period of time in accordance with one or more selected DT (e.g., ECG, MRI, etc.) and form a record having a corresponding DT which may be stored in the EMR 130.

These records of different types may then be further processed to, for example, determine the physiological trend status of the patient as will be discussed below.

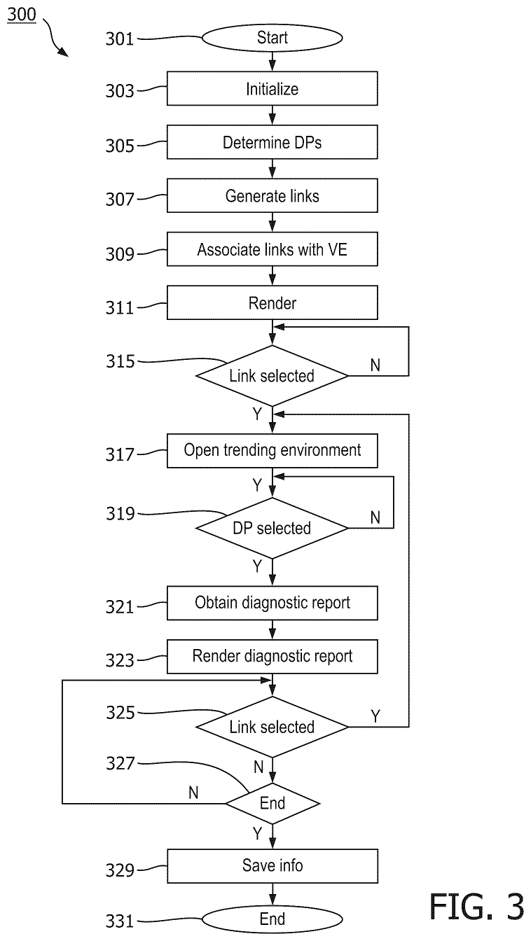

Operational processes performed by the system are now described with reference to FIG. 3 which illustratively shows a functional flow diagram performed by a process 300 in accordance with embodiments of the present system. The process 300 may be performed using one or more processors, computers, controllers, etc. communicating over a network and may obtain information from, and/or store information to, one or more memories which may be local and/or remote from each other. The process 300 may include one or more of the following acts. In accordance with embodiments of the present system, the acts of process 300 may be performed using a controller operating in accordance with embodiments of the present system. Further, one or more of these acts may be combined and/or separated into sub-acts, or performed serially or in parallel, as may be desired. Further, one or more of these acts may be skipped depending upon settings. For the sake of clarity, the process may be described with reference to a single SMU. However, without limitation, it should be understood that the process may employ a plurality of SMUs each of which may include a separate workflow such as a sub-workflow. In operation, the process 300 may start during act 301 and then proceed to act 303.

During act 303, the controller may be initialized by performing an initialization routine. During initialization, the process may load system setting information (SSI) and may set system settings in accordance with the SSI. For example, the process may obtain trend study information (TSI) for one or more trend studies (TSs) which may be performed by the system during the process. For example, the TSs may be selected by the default configuration (e.g., to run automatically or when one or more other TSs are performed) as may be set forth by the SSI and/or by the clinician (e.g., during performance of the process 300). The process may further obtain corresponding neural network models and/or algorithms from a memory of the system that may be employed by the process to determine trends in accordance with embodiments of the present system. The SSI may include trend study information (TSI) which may include, for example, instructions, settings, algorithms, and/or a date/range for a particular TS or for a plurality of TSs. For example, an ECG TS may determine trends using at least ECG records (e.g., EMR ECG records) while an MRI TS may determine trends using at least MRI records (e.g., MRI ECG records). The TSI may include information related to types of EMRs to be obtained for the corresponding study. Table 1 illustrates an exemplary TSI for an ECG TS that may be performed by the process to track a specific disease(s). Table 1 may be associated with for ECG type diagnostic TSs. The TSI may set forth operations to be performed to track one or more trends and to form a corresponding TS. Settings may be set and/or modified (e.g., at any time) by the clinician as may be desired and may be stored in the SSI for later use.

TABLE 1

| GRAPH | | | DMS | | |
|---|---|---|---|---|---|
| Type | DESCRIPTION | Perform | TYPE | Capture | Date/Range |
| LVH, | LVH, Sokolow Lyon and | Yes | ECG | End | Last 5 tests |
| Sokolow | Cornell equations. (may be | | | | |
| Lyon and | used to track patients on blood | | | | |
| Cornell | pressure medication and may | | | | |
| equations | also be used to track the | | | | |
| | evolution of Aortic Valve | | | | |
| | disease). | | | | |
| Heart Rate | Atrial and ventricular heart | Yes | ECG | End | Jan. 1, 2020- |
| (4 Graphs) | rate | | | | current |
| | PP, RR, and PR intervals | Yes | ECG | End | Jan. 1, 2020- |
| | | | | | current |
| | PR, QRS, QT intervals | Yes | ECG | End | Jan. 1, 2020- |
| | | | | | current |
| | QT and all forms of QTc | No | ECG | End | Jan. 1, 2020- |
| | (currently developed) | | | | current |
| Rhythm | Sinus rhythm with/without | Yes | ECG | End | Jan. 1, 2000- |
| (visually | ectopy, AF/AFL, etc.) | | | | Jan. 1, 2010 |
| represent | | | | | |
| findings | | | | | |
| of each | | | | | |
| study) | | | | | |
| Axis | Calculate | Yes | ECG | End | Age 16-20 |
| Ischemia | Graph of all ECG leads and | Yes | ECG | End | Jan. 1, 2000- |
| | their respective ST segment | | | | current |
| | variation from baseline | | | | |
| Hypertrophy | Right Atrial Enlargement/Left | Yes | ECG | End | Jan. 1, 2000- |
| | Atrial Enlargement, Right | | | | current |
| | Ventricular Hypertrophy/Left | | | | |
| | Ventricular Hypertrophy | | | | |
| | parameters | | | | |
| Heart | Graph/Table representing | No | ECG | End | Jan. 1, 2000- |
| Block | findings of heart block for each | | | | current |
| | study | | | | |
| Aortic | ECG Right bundle branch | Yes | ECG | End | Jan. 1, 2000- |
| valve | block trend, compared to | | | | current |
| disease | Ultrasound Ejection Fraction | | | | |
| | trend | | | | |

With reference to Table 1, the column entitled Graph indicates graphing operations to perform to track trends such as graph “Heart Rate” and may include first through fourth graphs that may be generated by the system and may include, for example: (1) atrial and ventricular heart rate; (2) PP, RR, and PR intervals; (3) PR, QRS, and QT intervals; and (4) QT and QTc, respectively. Each of these graphs may include data (e.g., illustrated as trendlines or other desired graphing obtained from corresponding ECG sensors and may be generated separately or together (e.g., on a single graph) and may be differentiated using, for example, different colors, depending upon system settings. For the sake of clarity, graphs may include trend lines and may be differentiated by color. However, it should be understood that other forms of graphing may be provided such as area charts, bar charts, etc. as may be selected by the system and/or clinician.

Depending upon settings, trends over different measurements (e.g., heartrate, QT, etc.) may be represented in the same or in separate graphs. For example, rendering trends over different measurements in different graphs, may aid in distinguishing between trends that may be related to different diagnosis. Conversely, rendering a single graph with combined trends may aid in recognizing trends related to a single diagnosis. In some embodiments, options may be provided for the clinician to select desired settings.

The column entitled “Perform” may indicate whether an operation is to be performed as indicated by a yes or no (or 0 and 1) and may be customized by the clinician. For example, all graphs indicated by a “yes” may be graphed except for those marked with a “no” such as QT and all forms of QTc as well as Heart block. This may simplify graphs to include desired information only. This may be customized on an enterprise level and/or by a corresponding clinician and stored for later use. For example, the SSI may set forth which STI is to be employed for each clinician.

The column entitled “DMS type” may indicate diagnostic medical study type(s) which are to be analyzed during the process such as ECGs in the present embodiments. However, it should be understood that other DMS types (such as: MRI, CT, Ultrasound, ICL, Pathology, etc.) may also be included and used to extract EMRs for analysis, if selected and/or available. If particular EMRs are not available the system may provide an indication of such depending upon system settings.

The column entitled “capture” may indicate a time at which corresponding data (e.g., to form a data point (DP)) is to be captured such as at end of corresponding graph as shown (or at other times at beginning, middle, etc. as may be desired). Thus, for example, the heart rate may be captured at a discrete time, such a such as at the end of a record (e.g., a corresponding ECG record) and may form a data point (DP) for a corresponding trend line. In some embodiments, the DPs may be determined by taking an average of the corresponding data over a desired period of time (e.g., 1 hour). Thus, if desired, this average may form a DP for HR.

The column entitled “Date/Range” may include a date(s) and/or date range(s) over which EMR data is to be analyzed (and thus collected). The Date/Range may be set by the system and/or clinician. When it is detected that the clinician has selected a Date/Range item, the system may render a calendar for the clinician to select desired days, dates, ranges, duration, etc. to be entered in that item (e.g., box). In some embodiments, the system may provide available dates, range(s), and/or available reports for selection. In some embodiments, the Date/Range may include an option to select individual reports such as first report for each year from 2001 through current, etc. In some embodiments, the representation of time can be distributed with even spacing between each date, or it can be spaced relative to actual time. For example, study dates in 1990, 1992, 2001, 2023 will have even spacing between data points, or the same dates could be displayed according to the actual calendar 1990 and 1992 will be close to each other with 2023 being relatively far away from 1990/1992.

It is further envisioned that clinicians may be provided with an option by the system to filter specific date ranges of studies. For example, patients such as premature babies may have daily ECGs, and as they age (e.g., over their lifetime) they might receive annual ECGs. Then, when these patients reach 20 years old, a clinician may desire to view the trend over a specific set date range, or ranges such as over an age range of 16-20 years old (e.g., as shown in the Axis row “Age 16-20”). Thus, options to select a date/range over specific time or patient (age) may be provided by the system and may be set/reset by the clinician as desired.

Once the TSI is set/reset it may be stored as SSI in a memory of the system for later use. It is envisioned that the SSI including the TSI may be customized at any time for a particular clinician and/or patient. For example, the clinician may select a trend graph and the system may generate and render a menu with which the clinician may interact to change settings of the TSI and thereafter the system may save these settings in the SSI and store then in a memory of the system for later use.

For the sake of clarity, it will be assumed that the SSI may correspond with a current clinician and/or patient. Accordingly, it is assumed that information identifying the clinician and/or patient (e.g., PI) has been provided to the system or may be provided during the present act. Accordingly, it may be assumed that a request for patient identification (PI) may be rendered and has been entered by the clinician in response. However, in some embodiments, the PI may be obtained automatically (e.g., during a patient scan in, etc.). In some embodiments, it is envisioned that the SSI may be selected independent of either the clinician (e.g., may be set for an enterprise such as a diagnostic center, a hospital, etc.) and/or patient.

After completing act 303, the process (e.g., though operation of the controller 120) may continue to act 305 where the process may determine DPs in accordance with the SSI for the current patient. Accordingly, the process may obtain EMR (e.g., selected EMR) for the current patient in accordance with SSI. This EMR may be limited to a date range (e.g., Jan. 1, 2000-current, ages 16-20, etc.) and diagnostic types (e.g., ECG, MRI, etc.), and sensory types (e.g., PP, RR, and PR intervals, etc.), etc. as may be set forth in the SSI. The process may then extract one or more DP from the (selected) EMR in accordance with the SSI (e.g., as set forth in the TSI) and these DPs may form one or more of the determined DPs. After completing act 305, the process may continue to act 307.

During act 307, the process may generate one or more links between the determined DPs and the corresponding EMR from which they were derived. These links may be associated with a reverse link back to the DP. For example, the EMR and the DP (derived from the corresponding EMR) may each be associated with links to each other. These links may be referred to as a reverse link pair (RLP). Thus, for each DP, the process may generate a link to the EMR from which the DP was derived and may generate a reverse link so as to optionally form an RLP. After completing act 307, the process may continue to act 309.

During act 309, the process may associate the determined one or more links (and/or RLP depending upon embodiments) with a current diagnostic viewing environment (VE) and/or worklist/inbox (WI) viewing environment (VE) for a desired diagnostic type (e.g., ECG in the present embodiments) for a current patient that may be rendered by the system on a rendering device of the system. After completing act 309, the process may continue to act 311.

During act 311, the process may render the current diagnostic viewing environment (VE) and/or worklist/inbox (WI) viewing environment (VE) with the associated determined one or more links. These links may be generated with the current diagnostic viewing environment (VE) and/or worklist/inbox (WI) viewing environment (VE) or may be superimposed thereon. The process may provide a workplace with which the clinician may interact with the diagnostic viewing environment (VE) and/or worklist/inbox (WI) viewing environment (VE) to, for example, select a file, a link, a graph, etc. For example, with reference to FIGS. 2A-2B, the clinician may interact with the ECG viewing environment (VE) 201 including its first and/or second EGC graphs 203 and 205 to select a desired graph and/or point or may interact with the worklist/inbox graph 203 to select a file, etc. For example, with reference to graphs 201 and 202, links may be generated and associated with the graph lines (or other elements as may be set by the SSI) of either of graphs 201 or 202. One or more of the DPs may have been derived from these graph lines depending upon system settings as may be set forth in the SSI. With reference to the ECG VE 201, links may be generated and associated with the listed files. Accordingly, clicking on these links (e.g., single or double clicking as may be set forth in the SSI) or on a data point (DP) may be considered selection of the corresponding link for the sake of clarity. Although links are discussed in the present embodiments, it is also envisioned that one or more DPs or portions of the screen may include hotspots which may be configured for hyperlinking or other activities such as GUI activities (e.g., mouseovers, etc.). Thus, the links may include hyperlinks situated in hotspots and which may activate GUI activities such as mouseovers or the like. In this regard, it is also envisioned that hovering over the links, may cause the system to open a corresponding linked pop-up window which may illustrate information such as trend graphs. After completing act 311, the process may continue to act 315.

During act 315, the process may determine whether the clinician has selected a link in the current viewing environment (VE) (e.g., see, FIGS. 2A-2C). Accordingly, if it is determined that the clinician has made a selection in the current VE, the process may continue to act 317. However, if it is determined that the clinician has not made a selection in the current VE, the process may repeat act 315. During this act, the process may determine whether one of more links generated by the system has been selected (e.g., by a clinician) from within the current VE.

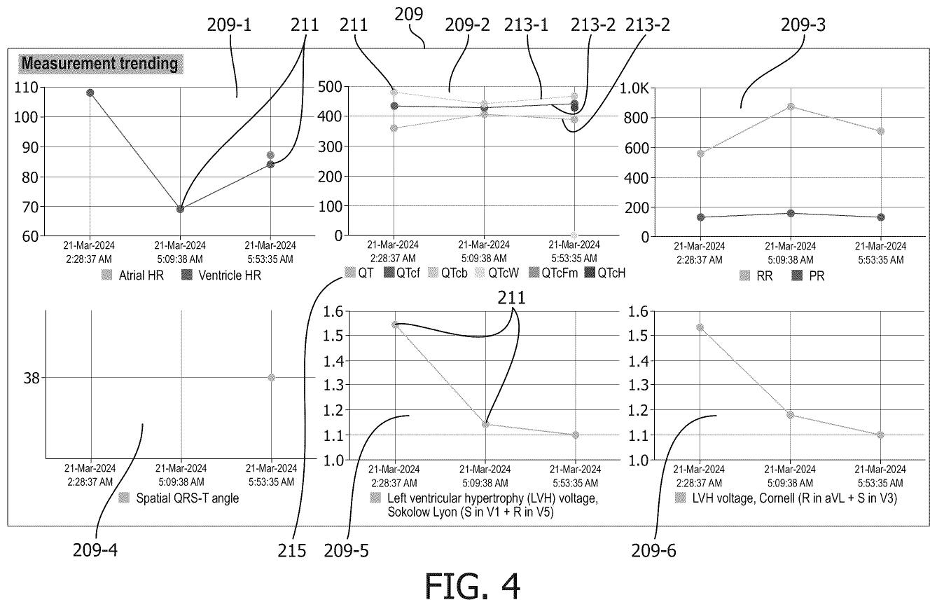

During act 317, the process may generate and render a trending environment (TE) in accordance with the selected link. Depending upon system settings, this TE may include a trending view (TV) graph (e.g., see, FIG. 2C, 209) which may include a single graph or a plurality of subgraphs, such as subgraphs 209-1 through 209-6 as illustrated. These graphs are illustrated in further detail with respect to FIG. 4 which is an illustration of a portion of the diagnostic viewing environment 209 of FIG. 2C in accordance with embodiments of the present system. The diagnostic viewing environment of the trend view graph 209, is a trending environment that may be launched by selecting links mapped to this environment from either of the Patient's diagnostic ECG viewing environment and worklist/inbox viewing environment. As shown multiple trends (e.g., trend lines as set forth by corresponding DPs 211) may be simultaneously rendered by the system in the diagnostic viewing environment trend view graph 209. Accordingly, a plurality of measurement trends using one or more charts may be rendered at a single time and viewed simultaneously by the clinician.

Each of the trend view subgraphs 209-1 through 209-6 (generally 209-x) may include one or more data points (DPs) 211 which may be graphically represented using any suitable charting method(s) such as line chart and may include a link that may correspond with the EMR from which the corresponding DPs were derived. Thus, the DPs may be mapped to the corresponding EMR from which they were derived and the corresponding EMR may be opened and/or rendered by selecting the DP (e.g., using any selection method such as by hovering, clicking, double clicking, etc., as may be set forth by the SSI). Each of the data point (DPs) 211 may be placed in a desired order such as in an order of time as shown. However, other orders are also envisioned and may be set by the system and/or user.

Subgraphs 209-x may be trend view graphs and may include corresponding titles, descriptions, and legends 215. Dates and/or report numbers from which corresponding data points (DPs) 211 were derived may be placed along the abscissa (e.g., X coordinate) and measurement values may be placed along the ordinate (e.g., Y coordinate) or each graph as may be set in the SSI. Although other settings are also envisioned. It is envisioned that some trend view graphs may include multiple trend lines such as trend view graph 209-2 in which first through third trend lines 213-1, 213-2, and 213-3, respectively, are illustrated and may be distinguished using different colors and/or line styles. One or more graphical control elements may be associated with one or more of the data points (DPs) 211. For example, a hotspot may be linked to a widget including information related to a report from which the data point (DP) and (the widget) maybe activated in response to a mouseover or other mapped action over the DP.

In some embodiments, it is envisioned that the trending environment (TE) including a trend view graph may be launched from a third party application such as an EMR or ECG management system using an iFrame. This is illustrated with reference to FIG. 5 which is an illustration 500 including portions of viewing environment 501 and a trend view graph 509 generated in accordance with embodiments of the present system. The trend view graph 509 may be similar to the trend view graph 209 and may be generated within an iFrame 511 and superimposed upon a current window of the viewing environment 501 which is a third party viewing application such as a diagnostic ECG viewing or the like. Similarly links to the trend view graph 509 may be inserted in the viewing environment using iFrames.

The process of generating and/or rendering the trending environment using the trend view graph may be referred to as launching or opening the trending environment.

In some embodiments, the process may compare values of the data points (DPs) with corresponding threshold values and may highlight a corresponding DP if the DP is less than or greater than the corresponding threshold value. Similarly, an absolute value of a slope generated between two or more serial data points (DPs) of the data points (DPs) may be compared to a corresponding threshold value and the absolute value of the slope is greater than the threshold value the process may highlight the slope when rendered for the convenience of the clinician. After completing act 317, the process may continue to act 319.

During act 319, the process may determine whether a data point (DP) is selected.

Accordingly, if it is determined that a DP is selected, the process may continue to act 321. If it is determined that a DP is not selected, the process may repeat act 319. The process may determine that a DP is selected when a link associated with the corresponding DP is selected (e.g., by hovering over, by single clicking, double clicking, etc., as may be set by the SSI). The DP may be selected from the trending environment via the trend view graph.

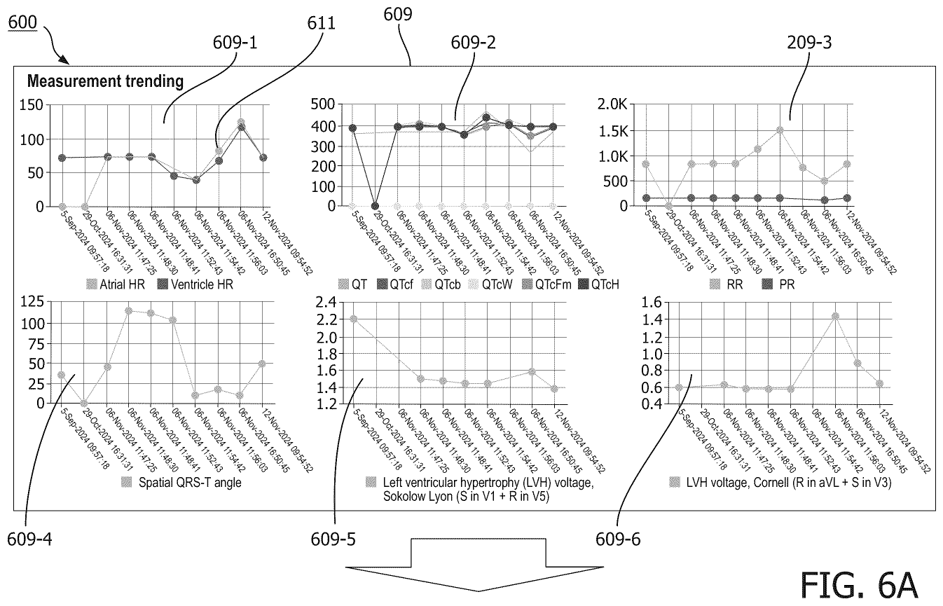

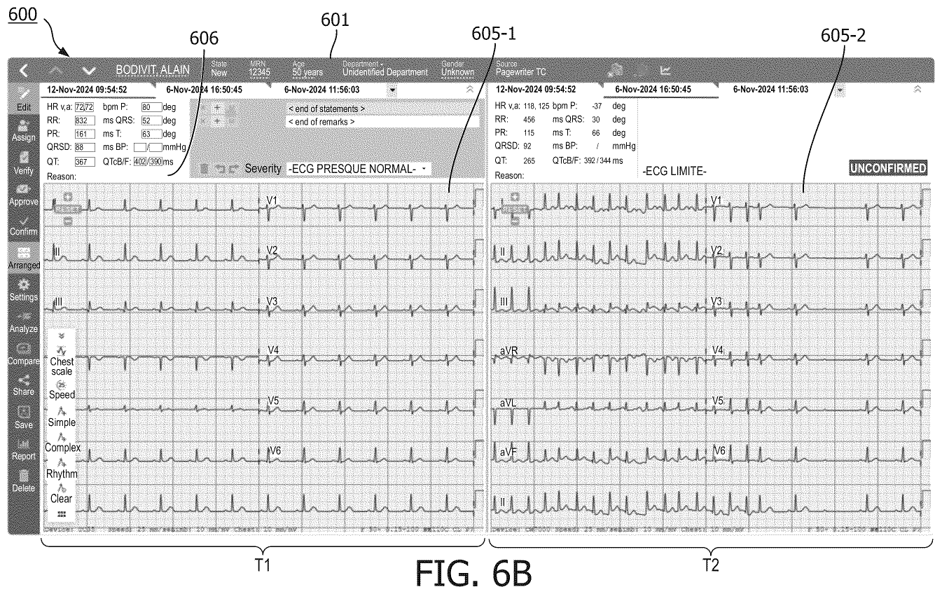

During act 321, the process may obtain a diagnostic report (e.g., an EMR in the present embodiments) corresponding to the data point (DP) that was determined to be selected (e.g., during act 319) from a memory of the system and may thereafter continue to act 323 where the process may be operative to render the corresponding diagnostic report (e.g., the EMR) that corresponds with the data point (DP) that was determined to be selected (e.g., during act 317). For example, FIGS. 6A-6B is an illustration 600 including portions of a diagnostic ECG viewing environment 601 and trending environment including a trend view (TV) graph 609 generated in accordance with embodiments of the present system. The ECG viewing environment (VE) 601 may include first and second EGC graphs 605-1 and 605-2 corresponding to ECG records (ECG scans) captured over two selected ranges of time (e.g., T1 and T2, respectively) and generated in accordance with embodiments of the present embodiments for a selected patient (e.g., the current patient). The ECG viewing environment may be similar to the ECG viewing environment 201.

The trend view environment may include information related to trend information obtained from selected EMRs of the current patient. The trending environment may include the trend view (TV) graph 609 which may be referred to as a trending view graph 609 and may include subgraphs 609-1 through 609-6 although other numbers of trending view subgraphs are also envisioned. The trending view graph 609 may be similar to the trending view graph 209 and may include one or more data points such as data point (DP) 611. Each of the DPs may include a reverse link of a reverse link pair (RLP) as discussed with reference FIG. 2C. Accordingly, each DPs of the DPs may be linked to the EMR from which it was derived. Accordingly, if it is determined that a DP has been has been selected (e.g., by the clinician (single or) double clicking on the data point 611, etc.), the process may obtain a diagnostic record, such as an EMR, linked to the DP. This diagnostic record is the diagnostic record from which the corresponding selected data point (DP) (e.g., 611) was derived. Once the corresponding diagnostic record is obtained, the process may associate links with data in the diagnostic record (e.g., EMR in the present embodiment) an may render this diagnostic record including the associated links on a rendering device of the system as a current viewing environment (VE) for the convenience of the clinician. These links may be similar to the links described with respect to the diagnostic ECG viewing environment 201 discussed above with reference to acts 307 and 309. This ECG

After completing act 323, the process may continue to act 325 where it may determine whether the clinician has selected a link in the current viewing environment (VE) (e.g., see, FIGS. 2A-2C and the corresponding description). Accordingly, if it is determined that the clinician has made a selection in the current VE, the process may continue to act 317. However, if it is determined that the clinician has not made a selection in the current VE, the process may continue to act 327.

During act 327, the process may determine whether to end. Accordingly, if it is determined to end, the process may continue to act 329 where the process may save information generated by the process and optionally other information such as selected EMRs in accordance with the SSI. However, if it is determined not to end, the process may repeat act 325. The process may determine to end, when for example, a timer has elapsed. This timer may be set to any value and may start to run at the beginning of the process or at some other desired time or event. In some embodiments, a menu item may be provided and selection thereof may indicate to the process to end. After completing act 329, the process may continue to act 331 where it may end.

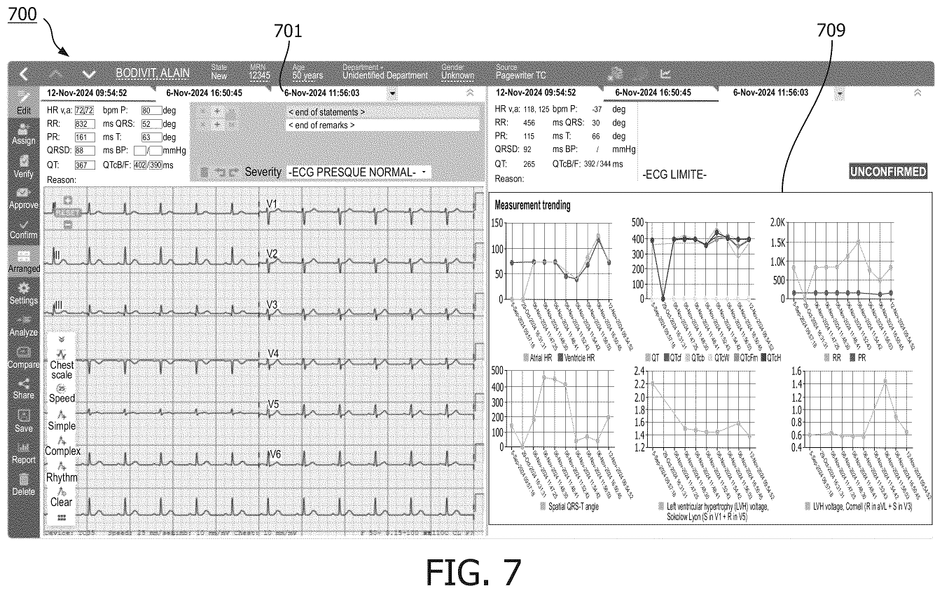

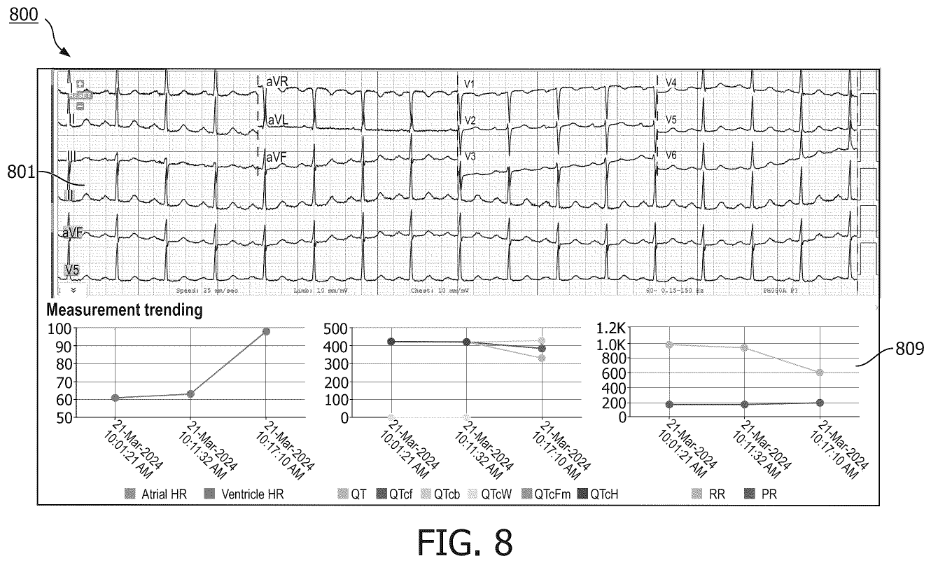

In accordance with embodiments of the present system, the trending environment may be viewed or rendered on a display in a side-by-side or stacked orientation with a current EMR as may be set forth in the SSI. This is illustrated with respect to FIG. 7 is an illustration 700 including portions of a diagnostic ECG viewing environment 701 and trending environment including a trend view (TV) graph 709 arranged in a side-by-side orientation generated in accordance with embodiments of the present system; and FIG. 8 which is an illustration 800 including portions of a diagnostic ECG viewing environment 801 and trending environment including a trend view (TV) graph 809 in a stacked view orientation generated in accordance with embodiments of the present system. With reference to FIGS. 7 and 8, the process may provide an interface such as a GUI with which the clinician may customize views as may be set forth in the SSI and stored in a memory of the system. Accordingly, a trending environment may be viewed on a single display next to, on top of or below current diagnostic data such as an ECG, etc. of the patient.

In accordance with embodiments of the present system detailed, patient level, physiological data, and/or like anatomical measurements, from each clinical study may be organized and archived into a database such as in a memory of the system for later retrieval and/or processing. It is envisioned that graphs may include individual data elements such as a patient's atrial heart rate which may be graphed over time. A graphical representation in ECG of average beats where the columns correspond to a recorded ECG and the rows correspond to ECG leads, may also be generated and rendered when requested. Trend lines may be added to graphs to help clinicians recognize trends. It is envisioned that an average trendline can be added to one or more graphs depending upon system settings as may be set forth in the SSI and/or a request by a clinician. Menu items may be provided for requesting and rendering desired information generated in accordance with embodiments of the present system. Multiple graphs may be added and available in a single view.

In accordance with embodiments of the present system clinicians may customize the graphs according to the needs of their diagnostic evaluation, choosing specific measurements to be represented on separate or the same graph. Accordingly, an interface may be provided with which the clinician may interact with to customize settings and these settings may be stored in the SSI for later use. When in a trending environment two or more studies may be selected and in response the system may perform a serial comparison algorithm across those studies. Results of the serial comparison study may be rendered for the convenience of the clinician.

With regard to data points (DPs), it is envisioned that in response to hovering over a data point (DP), the system may render the value of the data point (DP). For example, in a trendline with 15 data points (DP) (each of which may represent on of 15 clinical studies on different dates, etc.), the values of each of each of these data points (DPs) may be rendered in response to the system detecting that the when the user hovers over one point they will see its value (the data point for atrial heartrate on 2020 Dec. 20 shows the value of “120 bpm”). In accordance with embodiments of the present system, links may be provided at each data point (DP) and may be operative to render a value for the corresponding data point (DP) in response to detecting hovering over the data point (DP). In some embodiments, a clinician may select a data point (DP) (e.g., by clicking on the data point (DP) and the actual, full clinical full clinical evaluation (clinical study) from which the data point was derived may open and be rendered on the display. Accordingly, each data point (DP) may be associated with a hyperlink to the corresponding clinical study. It is further envisioned that embodiment of the present system may, in response to a clinician's request, may render multiple full studies to be viewed together, for example, in a traditional side-by-side or vertically-stacked perspectives.

In accordance with embodiments of the present system a trending view may be a dedicated view such as a pop-out. It is envisioned that trending views may be opened in a different browser tab and/or window. Further trending views may be embedded into different application(s). Options may be provided to save a customized trend view in the application and share the trend view with other users.

It is envisioned that when clinicians are in “other” applications they will be able to view trendlines using technologies like iFrame, Single-Sign-On, WebAPI, and API by launching the trending environment operative in accordance with embodiments of the present system.

Embodiments of the present system may be configured to share a link to a particular patient's trend screen with another person. Embodiments of the present system may save a trending report as a PDF, doc, or other filetype. It is further envisioned that embodiments of the present system may export discrete data elements of the trend screen in formats like csv, FHIR, XML, HL7 formatted text. These data elements may include for example: data points (DPs), trendlines, diagnostic statements, measurement names, a title of trend graph, graph axis labels, patient demographics, and study data coming from Health Level Seven (HL7) orders and an Admission, discharge, and transfer system (ADT).

Table 2 includes formulas for measurements:

TABLE 2

| TITLE | CALCULATION FORMULA |
|---|---|
| Heart Rate [Top Left] | This is the heart rate we can see in measurement area. The |
| | diagram will show both the Atrial HR and Ventricle HR if |
| | the data is available. |
| QT, and QTc [Top | QTC (Fridericia correction) = XML |
| Middle] | /interpretations/interpretation/globalmeasurements/qtcf |
| | QTC (Bazett correction) = XML |
| | /interpretations/interpretation/globalmeasurements/qtcb |
| RR interval & PR | The interval between two R wave, and the duration from the |
| interval [Top Right] | start of P wave to the end of R wave. |
| | RR = XML |
| | /interpretations/interpretation/globalmeasurements/rrint |
| | PR = XML |
| | /interpretations/interpretation/globalmeasurements/print |
| Spatial QRS-T angle | spatialqrstanglemax |
| [Bottom Left] | |
| Left ventricular | Abs(samp) in V1 + Abs(ramp) in V5 |
| hypertrophy (LVH) | |
| voltage, Sokolow Lyon | |
| (S in V1 + R in V5) | |
| [Bottom Middle] | |
| LVH voltage, Cornell | R in aVL + S in V3 |
| (R in aVL + S in V3) | |
| [Bottom Right] | |

Embodiments of the present system may combine the synthesis of historical biometric trends with information technology (IT) solutions such as Single Sign-on (SSO) and Clinical Context Management (CCOW) to allow Clinicians to launch directly into trending data without opening and logging into applications and searching for patients and prior diagnostic studies. Embodiments of the present system may provide for launching specific trend screens and seeing data in the trends without switching applications during use.

It is envisioned that to build graphs of biometric trends, data may be captured from various medical devices, organized in a database, and presented as graphs and other relevant views in a secure webpage. This webpage may be programmed to have the ability to be securely launched from other applications. Clinicians, while working in one application, like an EMR Application, a hospital information system (HIS), or a picture archiving and communication system (PACS), will be able to click a link, often on a word or icon, in their current application. This link may be associated to the specific trending webpage for a current patient. Clinicians may also be able to select specific points on the trends such as data points (DPs) and, in response may be provided with a more detailed clinical data associated with that point (or specific clinical study (such as CAT scan, X-ray, echocardiogram, electrocardiogram, stress test) that produced the data) from which the data point (DP) or other data points (DPs) may have been obtained. The clinician may view the biometric trends while making clinical decisions. This may be referred to as the clinician's clinical workflow.

Embodiments of the present system provide a system and method which may render diagnostic biometric trends across a patient's history in disparate systems which can enable efficient viewing of relevant trends from within a clinician's workspace. It is envisioned that embodiments of the present system may synthesize specific diagnostic trends into easily readable graphs which may then be rendered on a rendering device of the system for the convenience of the clinician. These graphs can be launched and viewed directly from the EMR or other applications, within an appropriate clinical context.

It is envisioned that during a diagnostic interrogation, clinicians may interact with embodiments of the present system which may render information related to a general patient history of the patient available from the EMR of the patient and may be informed when a patient has a long history of cardiology disease. During the diagnostic interrogation embodiments of the present system may provide relevant information such as trends of ECG data (for example increasing heart rate over time) which information may be easily accessed by a clinician by, for example, simply clicking on a link in the EMR, and in response, the system may render relevant trending information on a display of the system. Relevant trends from the patient's previous ultrasound studies such as, reduced Ejection Fraction, etc. may be determined and rendered on the display for the convenience of the clinician. Trend screens combining and synthesizing data from multiple diagnostic study types may also be rendered on the display within a current application of the clinician without logging in to one or more disparate systems. Trending graphs may inform a clinician as to where to concentrate on a particular study and/or which clinical protocols to employ.

Embodiments of the present system may generate and render one or more graphs trending biometric data, for example ECG, Ultrasound, hemodynamic, etc. and includes customizable methods for viewing, launching, and accessing the graphs from various locations.

Accordingly, embodiments of the present system may overcome disadvantages of existing systems and may provide access to, and the synthesis of, historical physiological data such as may be stored in EMRs and the like. Evolution in a patient's physiology is important to accurately diagnose several diseases. Embodiments of the present system may automatically fetch, analyze, and form trend information which may be employed to evaluate evolutionary change of the patient without the need to individually open several reports (e.g., in PDF format or the like), manually observe particular physiological measurements, and/or memorize visual observations, to mentally stitch together trends in the physiological measurements to determine if there is a concerning change in anatomy of the patient. In the process of manually performing this data synthesis there may be the need to login and access different information technology (IT) systems, logging in and out, to see the required data. Sometimes they need to memorize various physiological trends and coalesce them into a single entity because the combination of different trends gives insights into a particular disease. This becomes particularly difficult when physicians need to reference data in studies that are done with high frequency over time, like electrocardiogramatudies. They might need to evaluate 20-30 different studies taken over multiple years, memorize that data, and synthesize it for one diagnostic evaluation. This process can be difficult if not impossible to perform is error prone and time consuming which results in higher health care costs. Accordingly, embodiments of the present system overcome these disadvantages.

Further variations of the present system would readily occur to a person of ordinary skill in the art and are encompassed by the following claims.

Finally, the above discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art including using features that are described with regard to a given embodiment with other envisioned embodiments without departing from the broader and intended spirit and scope of the present system as set forth in the claims that follow. In addition, any section headings included herein are intended to facilitate a review but are not intended to limit the scope of the present system. In addition, the specification and drawings are to be regarded in an illustrative manner and are not intended to limit the scope of the appended claims.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function;
e) any of the disclosed elements may be comprised of hardware portions (e.g., including discrete and integrated electronic circuitry), software portions (e.g., computer programming), and any combination thereof;
f) hardware portions may be comprised of one or both of analog and digital portions;
g) any of the disclosed devices, features and/or portions thereof may be combined together or separated into further portions unless specifically stated otherwise;
h) no specific sequence of acts or steps is intended to be required unless specifically indicated; and
i) the term “plurality of” an element includes two or more of the claimed elements, and does not imply any particular range of number of elements; that is, a plurality of elements may be as few as two elements, and may include an immeasurable number of elements.

1. A clinical monitoring system for determining trends across medical studies, comprising:
at least one controller configured to:
generate a plurality of links each link associating a corresponding data point (DP) of a plurality of data points (DPs) with a medical record of a plurality of medical records from which the corresponding data point (DP) was derived;
render a medical viewing environment (VE) for a current diagnostic type, the medical viewing environment (VE) comprising at least one link associated with trend information;
determine whether the at least one link associated with the trend information has been selected;
open a trend environment comprising the trend information and having at least one trend graph comprising a plurality of corresponding datapoints (DPs) of the plurality of data points (DPs) when it is determined that the at least one link associated with the trend information has been selected;
determine whether a data point (DP) of the plurality of data points (DPs) is selected in the trend graph; and
render the medical record associated with the selected data point (DP) by a corresponding link of the plurality of links.

2. The clinical monitoring system of claim 1, wherein the current viewing environment comprises a diagnostic viewing environment (VE) or worklist/inbox (WI) viewing environment (VE) for a current diagnostic type.

3. The clinical monitoring system of claim 2, wherein the diagnostic type is selected from one of an Echocardiogram (ECG) scan, an Ultrasound scan, a Computed Tomography (CT) scan, a Magnetic Resonance Imaging (MRI) scan, and Interventional Catheterization Laboratory (Cath Lab or (ICL)) report, and a Pathology Laboratory Analyzer (Pathology) report.

4. The clinical monitoring system of claim 1, wherein the at least one controller is further configured to obtain the plurality of medical records in accordance with system setting information (SSI) from an electronic medical record (EMR) database.

5. The clinical monitoring system of claim 1, the at least one controller is further configured to obtain the plurality of medical records associated with a selected patient.

6. The clinical monitoring system of claim 5, wherein the at least one controller is further configured to extract the plurality of data points (DPs) from the plurality of medical records associated with the selected patient.

7. The clinical monitoring system of claim 6, wherein the at least one controller is further configured to extract each of the plurality of data points (DPs) over a period of time in accordance with the system setting information (SSI).

8. The clinical monitoring system of claim 7, wherein the at least one controller is further configured to include a reverse link in the rendered medical record, the reverse link associating the selected data point (DP) and the medical record from which the selected data point (DP) was derived.

9. The clinical monitoring system of claim 8, wherein the at least one controller is further configured to determine whether the reverse link was selected, and return to the trend environment when it is determined that the reverse link was selected.

10. The clinical monitoring system of claim 1, wherein the at least one controller is further configured to determine whether hovering is performed over a data point (DP) of the plurality of data points (DPs) and render a value of the corresponding data point (DP) of the plurality of data points (DPs) when it is determined that hovering is performed over a data point (DP).

11. A clinical monitoring system for determining trends across medical studies, comprising:
at least one controller configured to:
generate a plurality of links each link associating a corresponding data point (DP) of a plurality of data points (DPs) with a medical record of a plurality of medical records from which the corresponding data point (DP) was derived;
render a medical viewing environment (VE) for a current diagnostic type, the viewing environment (VE) comprising at least one link associated with trend information;
determine whether the at least one link associated with the trend information has been selected;
open a trend environment comprising the trend information and having at least one trend graph comprising a plurality of corresponding datapoints (DPs) of the plurality of data points (DPs) when it is determined that the at least one link associated with the trend information has been selected;
determine whether a data point (DP) of the plurality of data points (DPs) is selected in the trend graph; and
render the medical record from which the selected data point (DP) of the data points (DPs) was derived from the plurality of medical records when it is determined that the data point (DP) was selected.

12. The clinical monitoring system of claim 11, wherein the viewing environment comprises a diagnostic viewing environment (VE) or worklist/inbox (WI) viewing environment (VE) for a current diagnostic type.

13. The clinical monitoring system of claim 11, wherein the at least one controller is further configured to obtain the plurality of medical records in accordance with system setting information (SSI) from an electronic medical record (EMR) database.

14. The clinical monitoring system of claim 13, the at least one controller is further configured to obtain the plurality of medical records associated with a selected patient.

15. The clinical monitoring system of claim 11, wherein the at least one controller is further configured to extract the plurality of data points (DPs) from the plurality of medical records associated with the selected patient.

16. The clinical monitoring system of claim 15, wherein the at least one controller is further configured to extract each of the plurality of data points (DPs) over a period of time in accordance with the system setting information (SSI).

17. A clinical monitoring system for determining trends across medical studies, comprising:
at least one controller configured to:
obtain a plurality of medical records corresponding to a patient in accordance with system setting information;
generate a plurality of links each link associating a corresponding data point (DP) of a plurality of data points (DPs) with a medical record of a plurality of medical records from which the corresponding data point (DP) was derived;
render a medical viewing environment (VE) for a current diagnostic type, the medical viewing environment (VE) comprising at least one link associated with trend information;
determine whether the at least one link associated with the trend information has been selected;
open a trend environment comprising the trend information and having at least one trend graph comprising a plurality of corresponding datapoints (DPs) of the plurality of data points (DPs) when it is determined that the at least one link associated with the trend information has been selected;
determine whether a data point (DP) of the plurality of data points (DPs) is selected in the trend graph; and
render the medical record associated with the selected data point (DP) by a corresponding link of the plurality of links.

18. The clinical monitoring system of claim 17, wherein the at least one controller is further configured to extract the plurality of data points (DPs) from the plurality of medical records associated with the selected patient.

19. The clinical monitoring system of claim 18, wherein the at least one controller is further configured to extract each of the plurality of data points (DPs) over a period of time in accordance with the system setting information (SSI).

20. The clinical monitoring system of claim 17, wherein the at least one controller is further configured to include a reverse link in the rendered medical record, the reverse link associating the selected data point (DP) and the medical record from which the selected data point (DP) was derived.
